(19) United States
(12) Patent Application Publication
(10) Pub. No.: US 2024/0337479 A1
(43) Pub. Date: Oct. 10, 2024

(54) Universal Photonics Tomography

(71) Applicant: THE REGENTS OF THE UNIVERSITY OF CALIFORNIA, Oakland, CA (US)

(72) Inventors: Andrew GRIECO, La Jolla, CA (US); Yeshaiahu FAINMAN, San Diego, CA (US); Prabhav GAUR, La Jolla, CA (US); Naif ALSHAMRANI, Riyadh (SA); Dhaifallah ALMUTAIRI, Riyadh (SA)

(21) Appl. No.: 18/579,312

(22) PCT Filed: Jul. 15, 2022
(86) PCT No.: PCT/US2022/037311

§ 371 Date: Jan. 12, 2024

Related U.S. Application Data
(60) Provisional application No. 63/222,368, filed on Jul. 15, 2021.

(51) Int. Cl. G01B 9/02091; G01B 9/02004; G01S 17/89

(52) U.S. Cl. CPC G01B 9/02091; G01B 9/02004; G01S 17/89

(57) ABSTRACT

A universal photonics framework is provided for improving axial resolution or depth measurement within an imaging system by incorporating phase modulation in combination with iterative scanning. Axial resolution and depth in an optical coherence tomography system are used to image surfaces of an object by scanning surfaces of the object with a tunable narrowband laser source and interferometer while applying phase modulation. The resulting interferogram is used to provide positional information for the surfaces.

### RELATED APPLICATIONS

This application claims the benefit of the priority of Provisional Application No. 63/222,368, filed Jul. 15, 2021, which is incorporated herein by reference in its entirety.

### FIELD OF THE INVENTION

The present invention relates to a system and method for performing tomography and more particularly to a method and system that uses a combination of an interferometer and a tunable delay line to generate a tomographic map.

### BACKGROUND

“Tomography” generally is the process of generating an image by sections using some form of penetrating electromagnetic wave. Coherent signal processing is a powerful tool for real time 3D imaging of objects at distances ranging from a few hundred microns to several hundred meters with corresponding resolutions. One application of real-time 3D imaging, Optical Coherence Tomography (OCT), is a well-developed imaging technique for objects at short distances with micron level resolution, hence is useful for various biomedical applications. OCT has two different forms: Time Domain OCT (TD-OCT) and Fourier Domain OCT (FD-OCT). The FD-OCT has been implemented exploiting two different approaches; the first one being Spectral Domain (SD-OCT), which utilizes a broadband source with spectrum analyzer. The second approach is the Swept Source (SS-OCT), which utilizes a tunable laser source combined with a photodetector. Among the different implementations, SS-OCT is the most promising, and can provide axial resolutions of 5 μm and depth information up to a few millimeters. Other variants of OCT such as Doppler OCT also exist for specialized applications where velocity measurement is also required.

For measurements of 3D objects at long distances ranging from a few meters to kilometers, a Light Detection and Ranging (LiDAR) technique is employed using a modulated source and a photodetector. LiDAR has a wide range of applications including, for example, surveying, forestry, atmospheric physics, and autonomous vehicles. The most common scheme to implement LiDAR is by measuring time of flight (TOF) of pulsed lasers. A more recently developed technique is frequency modulated continuous wave LiDAR (FMCW LiDAR) that uses a frequency chirp. The chirped signal is transmitted to the object and its replica is made to interfere with the returned signal that was reflected from the object. The beat frequency is then used to determine the distance to the object. It is worth noting that the technique of SS-OCT and FMCW LiDAR resemble each other in terms of their use of frequency sweep and measure distances using coherent detection. The difference arises from the manner of frequency sweep. In SS-OCT, a particular frequency interferes with itself, while in FMCW, LiDAR different frequencies can interfere with each other due to time lag.

According to conventional understanding, improvements to these technologies based on hardware have reached the point of diminishing returns. Research has consequently shifted to alternative methods, such as superior processing algorithms, and complex modulation/detection schemes, in a bid to improve the resolution and depth performance.

Additional challenges to optimization of optical imaging and transmission systems arise from waveguide losses due to scattering. The problem of unwanted scattering from tapering the parameters of transmission lines and waveguides was recognized very early in the development of the theory of electromagnetism, over a century ago. In the intervening years, numerous attempts have been made to identify the optimal adiabatic taper profile to minimize scattering loss. The need for a solution is only growing due in large part to the current renaissance of integrated photonics. The ultimate limit of system-on-a-chip devices is determined by the packing density of their components and interconnects, many of which have been miniaturized to the point that waveguide bending loss has become prohibitive. Future improvement will require optimal waveguide transitions to minimize excess loss. Additionally, due to their inherently broadband nature, adiabatic components are of particular interest to applications where this is required, such as spectroscopy.

### SUMMARY

According to the inventive approach, limitations in the prior art methods can be addressed by viewing these methods as part of a more general universal framework: a coherent interferometer that has the capability of optical modulation in different sections of the system. The prevailing techniques can all be viewed as special cases depending on the source, modulation format, and detection procedure. In one case, a laser, quadratic phase modulation and fast photodetector will implement FMCW LiDAR, whereas a frequency sweep and a slow photodetector becomes SS-OCT. A key benefit of this universal framework, which is referred to herein as “Universal Photonics Tomography” or “UPT”, is that it can enable formulation of novel reconfigurable functionalities and capabilities to these existing techniques. In one example, the phase modulator in OCT can be exploited to scan multiple times and can be used to detect objects over longer distances by changing the resolution and depth parameters of the tomography system. These parameters are a direct consequence of Nyquist criterion with length (or time) and frequency forming Fourier pairs.

Unlike conventional tomography methods that rely on broadband sources, implementations of the present approach use a single frequency (continuous wave) source. This provides a number of advantages, including: (1) samples with limited transparency windows no longer degrade the measurement resolution; (2) higher order material dispersion in the permeability and permittivity do not need to be estimated, which results in a more precise measurement; (3) the resolution and sampling rate are determined by the tunable delay line properties (rather than the properties of a broadband source), which are much more favorable; (4) single frequency devices are more amenable to chip-scale integration and miniaturization than broadband devices; and (5) single frequency tomography provides more useful information about the sample than does broadband tomography. For example, it can be used to provide information about the density of specific molecules in the sample. In broadband tomography information about specific frequencies gets washed out by the presence of so many other frequencies.

The inventive scheme provides a novel approach based on the use of phase modulation combined with multirate signal processing to collect positional information of objects beyond the Nyquist limits. Depending on the location of the phase modulator in the system, and associated modulation scheme, we can improve the axial resolution or the maximum measurement distance (unambiguous range). Using the framework of UPT, different types of sources, detectors, modulation devices, modulation schemes and digital signal processing can be combined to revolutionize the prevalent coherent tomography systems.

In one aspect of the invention, a method for determining axial resolution or depth in an optical coherence tomography system configured for imaging an object having one or more surfaces includes: scanning the one or more surfaces by projecting light from a tunable narrowband laser source into an interferometer to generate an interferogram while applying phase modulation to the projected light; and applying multirate signal processing to the interferogram to determine positional information for the one or more surfaces of the object. In some embodiments, applying phase modulation includes inserting a signal generator into a sample arm of the interferometer to apply phase modulation that is slow compared to the time taken to measure a single frequency. In other embodiments, applying phase modulation includes inserting a signal generator immediately downstream of the laser source to apply fast modulation to increase a maximum unambiguous range, wherein the fast modulation repeats after every sweep frequency. The method may further include repeating scanning and applying for multiple iterations.

In some embodiments, applying multirate signal processing includes defining multiple channels within the interferogram and combining the multiple channels in a frequency domain to increase time domain resolution. In other embodiments, applying multirate signal processing comprises defining multiple channels within the interferogram and interleaving the multiple channels to increase frequency resolution. In another aspect of the invention, a method for measuring one or more of axial resolution and depth of an object using an optical imaging system includes: applying phase modulation while scanning the object by projecting light from a tunable narrowband laser source into an interferometer to generate an interferogram, wherein the phase modulation changes resolution and depth parameters within the imaging system; and applying multirate signal processing to the interferogram to determine positional information for the object.

In some embodiments, applying phase modulation includes inserting a signal generator into a sample arm of the interferometer to apply phase modulation that is slow compared to the time taken to measure a single frequency. In such embodiments, the optical imaging system may be a swept source optical coherence tomography (SS-OCT) system. In other embodiments, applying phase modulation includes inserting a signal generator immediately downstream of the laser source to apply fast modulation to increase a maximum unambiguous range, wherein the fast modulation repeats after every sweep frequency. In such embodiments, the optical imaging system may be a Light Detection and Ranging (LiDAR) system. The method may further include repeating scanning and applying for multiple iterations.

In some embodiments, applying multirate signal processing includes defining multiple channels within the interferogram and combining the multiple channels in a frequency domain to increase time domain resolution. In such embodiments, the optical imaging system may be a swept source optical coherence tomography (SS-OCT) system. In other embodiments, applying multirate signal processing comprises defining multiple channels within the interferogram and interleaving the multiple channels to increase frequency resolution. In such embodiments, the optical imaging system may be a Light Detection and Ranging (LiDAR) system.

In yet another aspect of the invention, an assembly for determining axial resolution or depth in an optical coherence tomography system configured for imaging an object having one or more surfaces includes: a tunable narrowband laser source; an interferometer configured to generate an interferogram at a detector using light from the laser source; a phase modulator inserted within an arm of the interferometer; and a multirate filter bank configured for processing the interferogram to determine positional information for the object. In some embodiments, the phase modulator may be inserted into a sample arm of the interferometer, where the phase modulator is configured to apply slow modulation to the light to improve axial resolution in a length domain. In such embodiments, the optical imaging system may be a swept source optical coherence tomography (SS-OCT) system.

In other embodiments, the phase modulator may be inserted downstream of the laser source, where the phase modulator is configured to apply fast modulation to the light to increase a maximum unambiguous range of detection. In such embodiments, the optical imaging system may be a Light Detection and Ranging (LiDAR) system. In some embodiments, the multirate filter bank may be configured to define multiple channels within the interferogram and combine the multiple channels in a frequency domain to increase time domain resolution. The optical imaging system may be a swept source optical coherence tomography (SS-OCT) system In other embodiments, the multirate filter bank may be configured to define multiple channels within the interferogram and interleave the multiple channels to increase frequency resolution. In such embodiments, the optical imaging system may be a Light Detection and Ranging (LiDAR) system.

It is commonly recognized that waveguide tapering is important, however, there has been little rigorous analysis of the problem. What analysis has been done is typically limited in applicability to certain modes or material systems. In some reports, black box inverse design has been used to devise tapers, however, this approach does not produce any insight into what makes the tapers work, or how to extend them to other different contexts. Since the algorithms are statistical in nature, it is impossible to prove that such tapers are optimal.

The most rigorous prior art is known as the Milton-Burns taper (A. F. Milton and W. K. Burns, “Mode Coupling in Optical Waveguide Horns,” IEEE Journal of Quantum Electronics, Vols. QE-13, pp. 828-835, 1977.). Notably, this approach is limited to the interaction of the two lowest order modes, and there is no reason given to believe that it can be applied in different material systems or geometries.

The solutions provided by the inventive scheme are achieved via the use of a new method of determining the way to taper optical properties of waveguides while minimizing unwanted scattering. Common applications include matching mode profiles at photonic coupler inputs/outputs and bending waveguides to route light around a photonic chip. However, the derivation uses coupled mode theory, which is a very general formalism able to describe most physical phenomenon. Consequently, the method is applicable far beyond the context of optical waveguides in which it was originally conceived.

Conventional grating designs often relay on partially etched gratings or binary blazed gratings in order to enhance the coupling efficiency while attempting to reduce the reflected light. However, the impedance mismatch in such designs remains as an issue. The existing approaches include partially etched gratings (two step etch grating), one step etch grating, and binary blazed grating.

The inventive scheme addresses these issues using a novel approach to suppress reflections that frequently occur due to impedance mismatch during the coupling of light into and out of a photonic chip through integrated optical couplers (I/O's). The design is a conjunction of binary gratings and metamaterial structures (tapers). The inventive approach involves the use of metamaterial structure (tapers) to ensure the adiabatic transition of the refractive index (n), thus resulting in an impedance matching grating.

### BRIEF DESCRIPTION OF THE DRAWINGS

FIG. 1 is a schematic of a sample with arbitrary index profile partitioned into sections of equal time delay.

FIG. 2 is a schematic illustrating the tomography method according to an embodiment of the invention.

FIGS. 3A-3D illustrate different case of UPT, where FIG. 3A provides an exemplary test set-up without phase modulators (base case); FIG. 3B illustrates a first case with a phase modulator for slow modulation; FIG. 3C illustrates a second case with a phase modulator for fast modulation; FIG. 3D is a schematic for the working of UPT and the required post-processing in a filter bank form; FIG. 3E illustrates reconstruction signals formed by combining two channels (M=2) in the frequency domain for the cases of FIGS. 3B and 3C.

FIG. 4A-4C illustrate an experimental demonstration of UPT without modulators (base case), where FIG. 4A shows the test structure of a microscope slide and a mirror;

FIG. 4B provides the measured interference pattern on the photodetector as a function of frequency sweep; and FIG. 4C is the Fourier transform (FT) of the interference pattern.

FIGS. 5A-5E provide results using the inventive UPT approach for increasing axial resolution in a first case, where FIG. 4a shows the test structure of a microscope slide and a mirror; FIG. 5B shows the measured interference pattern of the unmodulated channel 0 and modulated channel 1 as function of frequency; FIG. 5C is a schematic of the linear modulation given to Channel 1 (Ch 1); FIG. 5D provides the Fourier transform (FT) of Ch 0 (upper curve) and Ch 1 ((lower curve); and FIG. 5E shows the synthesized distance estimation of the objects by combining both the channels as part of a lazy-filter bank. The resulting curve has twice better length resolution compared to the ones detected in the individual channels.

FIG. 6A is a schematic depicting the voltage applied to phase modulation as laser frequency is tuned; FIG. 6B shows the Fourier transform of the measured power when phase modulator is given 50 MHz sinusoidal signal at channel 0 (upper curve) and 80 MHz sinusoidal signal at channel 1 (lower curve); FIG. 6C shows the Fourier transform of the measured power when a mirror is actually placed in the aliased position of the original mirror; FIG. 6D shows the synthesized signal by combining the two channels and passing them through the synthesis filters.

FIG. 7 illustrates an example of optical I/O couplers with the impedance matching structure according to an embodiment of the invention.

FIG. 8 shows simulated spectral S parameters of optical I/O couplers with (lower) and without (upper) the impedance matching structure.

### DETAILED DESCRIPTION OF EMBODIMENTS

The following describes a new method for analyzing coherent tomography systems, in which conventional methods such as Optical Coherence Tomography (OCT) and Light Detection and Ranging (LiDAR) may be viewed as special cases. This general method, called “Universal Photonics Tomography” or “UPT”, can achieve significant hardware-based performance gains such as increased axial resolution and maximum depth measurement without increasing the signal bandwidth and frequency resolution of tunable laser. Unlike previous methods that require broadband sources, the inventive approach operates using a narrowband source. This is a major development that enables novel applications that are not possible with existing broadband methods. For example, since chemicals have narrow absorption bands, this technique can probe chemical concentration tomography. Solutions enabled by the inventive method include, but are not limited to, biomedical applications such as conventional tomography and tomography with molecular resolution, integrated photonics, and input/output couplers for wide variety of telecommunication and remote sensing applications.

Use of the inventive UPT enables formulation of novel reconfigurable functionalities and capabilities to existing techniques. In one example, the phase modulator in OCT can be exploited to scan multiple times and can be used to detect objects over longer distances by changing the resolution and depth parameters of the tomography system. These parameters are a direct consequence of Nyquist criterion with length (or time) and frequency forming Fourier pairs. They determine the limitations and effective cost of the system, and their relations are given by Eq. (1), where the axial resolution (lₒ) is mainly determined by the bandwidth (B) of laser sweep while the maximum distance (L) by the frequency resolution (υₒ).

$${l}_{o} = c / B ; L = c / 2 {υ}_{o} \qquad ( 1 )$$

Nearby object imaging is limited by the axial resolution lₒ (determined by the optical bandwidth) and far object imaging is limited by maximum distance L (determined by the frequency resolution). This tomography system is mathematically developed from first principles, showing how the fundamental resolution and depth limitations can be pushed using phase modulation and multirate filter bank interpretation.

Multirate filter banks are sets of filters, decimators, and interpolators used widely in conventional digital systems. Usually, decimators downsample the signal after passing through analysis filters. This compressed information is stored or transmitted via a channel. On the other end of channel, the signal is interpolated or upsampled and passed through synthesis filters to retrieve the original information. The process of downsampling means decreasing the resolution of system which is similar to an undersampled tomography system. The tomography systems are also discrete, and analog filters can be implemented by phase modulation of the optical carrier signal and by digital processing after detection. Hence, the imaging system can be considered as multirate filter bank with each scanning cycle representing a single channel and carrying object information in a compressed form. Here, a 2-channel filter bank implementation is demonstrated, resulting in a twofold improvement in both length and frequency resolution of the tomography system. Using this scheme, both near and far objects as well as their density profiles can be measured with improved parameters, providing improved versatility relative to conventional approaches.

The inventive approach employs two basic steps: (1) determine the electric field that is reflected from the sample as a function of time; and (2) use this time dependent reflection coefficient to determine the inner structure of the sample.

Table 1 provides a listing of the mathematical conventions and parameters employed throughout the written description:

TABLE 1

| | |
|---|---|
| I, S, | Used as subscripts, these indicate “input”, “sample”, |
| R, D | reference” and detector”, respectively |
| I | intensity |
| F | normalized electric field amplitude = \sqrt{I} |
| ω | frequency |
| τ | time |
| β | $effective wavenumber = n \frac{2 π}{λ}$ |
| n | refractive index |
| C_{1,2} | couple cross/through power split |
| L | interferometer arm length |
| ϕ | phase |
| r | sample reflection coefficient |
| t | transmission coefficient |
| ϕ_{I} | input field phase |
| ϕ_{T} | tunable phase shift |
| ϕ_{S} | sample reflection phase |
| N | number of Nyquist sampling points |
| Δt | time step resolution |
| Δf_{eff} | frequency step resolution |
| Δz | depth measurement resolution |

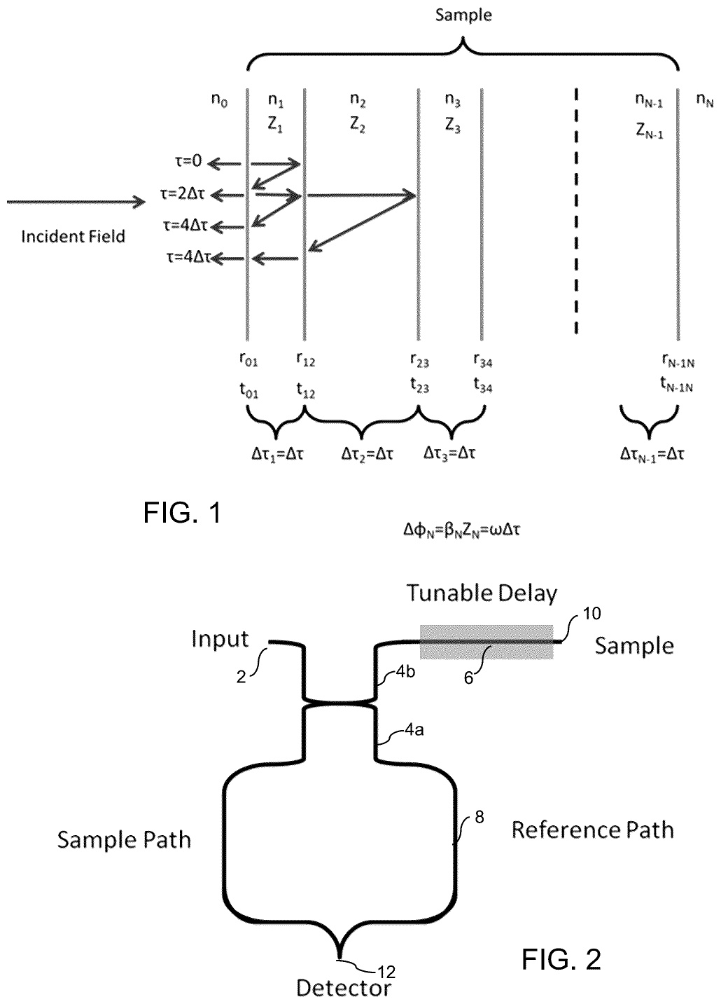

The method begins with development of a model of reflection from a sample with a general refractive index profile. For the derivation, a transparent sample is assumed for simplicity, however, it should be noted that loss is trivial to include in the model. To create the model, consider the sample profile partitioned into sections of equal time delay as shown in FIG. 1. If the time delay is small, the length of each partition will also be small, and the model will be a good approximation of an arbitrary sample.

The reflected field as a function of time may be written by adding the reflections from each of the layers. Note that as time passes, the light will bounce between multiple interfaces.

$${F}_{reflected} ( 0 ) = {r}_{01} {F}_{I} \qquad ( 2 )$$

$${F}_{reflected} ( 2 \cdot Δτ ) = {t}_{01} {r}_{12} {t}_{01} {F}_{I} exp ( i 2 ωΔτ )$$

$${F}_{reflected} ( 4 \cdot Δτ ) = [ {t}_{01} {r}_{12} {r}_{01} {r}_{12} {t}_{01} + {t}_{01} {t}_{12} {t}_{23} {t}_{12} {t}_{01} ] {F}_{I} exp ( i 4 ωΔτ )$$

$${F}_{reflected} ( 6 \cdot Δτ ) = [ \begin{matrix}{t}_{01} {r}_{12} {r}_{01} {r}_{12} {r}_{01} {r}_{12} {t}_{01} + {t}_{01} {r}_{12} {r}_{01} {r}_{23} {t}_{12} {t}_{01} + \\ {t}_{01} {t}_{12} {t}_{23} {r}_{34} {t}_{23} {t}_{12} {t}_{01}\end{matrix} ] {F}_{I} exp ( i 6 ωΔτ )$$

$${F}_{reflected} ( 8 \cdot Δτ ) = …$$

The key observation that allows the index profile to be calculated is that the earlier reflections are simple and allow the refractive index data to be inferred in such a way that the more complicated later reflections can be sorted out. For example, the earliest reflection depends only on the reflection coefficient of the first interface, which allows refractive index m to be determined given that no is known (no is the refractive index of the material in which the sample is immersed, generally air or water, so this is a fair assumption). Similarly, measurement of the combined with the knowledge from the first reflection allows the determination of m. This process can be extended to each subsequent reflection, allowing characterization of the entire sample.

The inventive method of tomography therefore hinges on finding the time dependent total reflected field. This has the general form below (including the continuous time limit):

$${{F}_{reflected} ( N Δ τ ) = a ( N Δ τ ) exp ( iN Δτω ) {F}_{I} \\ τ = N Δτ \vert}_{lim Δ τ \to 0} \\ {F}_{reflected} ( τ ) = a ( τ ) exp ( i τω ) {F}_{I} \qquad ( 3 )$$

A simple diagram of the general principles of how a device is arranged is provided in FIG. 2. Light from input 2 is split into two paths which are subsequently interfered. The first path 4a leads through a reference length. The second path 4b leads through a tunable delay line 6 and into the sample of interest 16. Light reflected from the sample 10 is then redirected to the detector 12 where it interferes with light from the reference path 8. When the time delay is tuned, an interferogram is created. When the paths are described mathematically, it can be shown that the interferogram is the Fourier transform of the reflected light as a function of sample depth. This is the tomography of the sample 10. Scanning the beam across the sample 10 can thus be used to create a 3D image of the sample density.

For measurement of the total reflected field, the field evolution along each path is calculated using the complex index notation. The input light accumulates a phase shift according to the paths that it travels. This is trivial to write for each path except for the light reflected from the sample. The general form for the total reflected light (as derived in the previous section) is as follows:

$${F}_{reflected} ( τ ) = a ( τ ) exp ( i ωτ ) {F}_{I} \qquad ( 4 )$$

Note that due to the complex exponential form that the reflection coefficient is a real number.

Consequently, the fields in the interferometer are as follows:

$${I}_{I} = {\vert {F}_{I} exp ( i {ϕ}_{I} ) \vert}^{2} \\ {F}_{R} = {F}_{I} exp ( i {ϕ}_{I} ) \sqrt{{C}_{1}} exp ( i k {L}_{R} ) = {F}_{I} \sqrt{{C}_{1}} exp ( i {ϕ}_{I} + i k {L}_{R} ) \\ {F}_{S} = {F}_{I} \sqrt{{C}_{1}} \sqrt{{C}_{2}} exp ( i {ϕ}_{I} + i k {L}_{S} ) \{ {\int }_{0}^{∞} a ( τ ) exp [ i ωτ + 2 i {ϕ}_{T} ( τ ) ] d τ \} \qquad ( 5 )$$

This leads to an intensity at the detector 12 of:

$$\begin{matrix}{I}_{D} ( k ) = {\vert {F}_{R} + {F}_{s} \vert}^{2} = [ {F}_{R} + {F}_{S} ] [ {F}_{R}^{*} + {F}_{S}^{*} ] = \\ {\underbrace{{F}_{R} {F}_{R}^{*} + {F}_{S} {F}_{S}^{*}}}_{DC Component} + {\underbrace{{F}_{R} {F}_{S}^{*} + {F}_{S} {F}_{R}^{*}}}_{Interference Term}\end{matrix} \} \qquad ( 6 )$$

This will appear as an interferogram with the interference term oscillating around a constant level set by the DC component. The interference term contains the important information.

$${I}_{D , Interference} = {F}_{R} {F}_{S}^{*} + {F}_{S} {F}_{R}^{*} = \begin{matrix}{F}_{I}^{2} {C}_{1} \sqrt{{C}_{2}} exp [ ik ( {L}_{R} - {L}_{S} ) ] \{ {\int }_{0}^{∞} a ( τ ) exp [ - i ω τ - 2 i {ϕ}_{T} ( τ ) ] d τ \} + \\ {F}_{I}^{2} {C}_{1} \sqrt{{C}_{2}} exp [ - ik ( {L}_{R} - {L}_{S} ) ] \{ {\int }_{0}^{∞} a ( τ ) exp [ i ω τ + 2 i {ϕ}_{T} ( τ ) ] d τ \}\end{matrix} \} \qquad ( 7 )$$

Making the trivial assumption that the interferometer sample and reference arms are equal in length, the interference term can be reduced to:

$$\begin{matrix}{I}_{D , Interference} = {F}_{I}^{2} {C}_{1} \sqrt{{C}_{2}} \{ {\int }_{0}^{∞} a ( τ ) exp [ - i ωτ - 2 i {ϕ}_{T} ( τ ) ] d τ \} + \\ {F}_{I}^{2} {C}_{1} \sqrt{{C}_{2}} \{ {\int }_{0}^{∞} a ( τ ) exp [ i ωτ + 2 i {ϕ}_{T} ( τ ) ] d τ \}\end{matrix} \} \qquad ( 8 )$$

Next, reverse the sign of the integration variable in one of the integrals.

$$\begin{matrix}{I}_{D , Interference} = - {F}_{I}^{2} {C}_{1} \sqrt{{C}_{2}} \{ {\int }_{0}^{∞} a ( τ ) exp [ - i ωτ - 2 i {ϕ}_{T} ( τ ) ] d τ \} + \\ {F}_{I}^{2} {C}_{1} \sqrt{{C}_{2}} \{ {\int }_{0}^{∞} a ( τ ) exp [ i ωτ + 2 i {ϕ}_{T} ( τ ) ] d τ \}\end{matrix} \} \qquad ( 9 )$$

Rewriting the reversed integral in terms of a dummy variable causes the limit to go from negative infinity to zero (i.e., covers the part of the integration domain that is not covered by the untouched integral). The tunable phase can be defined as a first order function.

$$\begin{matrix}{I}_{D , Interference} = {F}_{I}^{2} {C}_{1} \sqrt{{C}_{2}} \{ {\int }_{- ∞}^{0} a ( τ ) exp [ - i ω ( - τ ) - 2 i {ϕ}_{T} ( - τ ) ] d τ \} + \\ \begin{matrix}{F}_{I}^{2} {C}_{1} \sqrt{{C}_{2}} \{ {\int }_{0}^{∞} a ( τ ) exp [ i ωτ + 2 i {ϕ}_{T} ( τ ) ] d τ \} = \\ \begin{matrix}{F}_{I}^{2} {C}_{1} \sqrt{{C}_{2}} \{ {\int }_{- ∞}^{0} a ( - τ ) exp [ i ωτ + 2 i \frac{\partial {ϕ}_{T} ( τ )}{\partial τ} τ ] d τ \} + \\ {F}_{I}^{2} {C}_{1} \sqrt{{C}_{2}} \{ {\int }_{0}^{∞} a ( τ ) exp [ i ωτ + 2 i \frac{\partial {ϕ}_{T} ( τ )}{\partial τ} τ ] d τ \} \\ {ϕ}_{T} ( τ ) = \frac{\partial {ϕ}_{T} ( τ )}{\partial τ} τ\end{matrix}\end{matrix}\end{matrix} \} \qquad ( 10 )$$

Finally, combine the integrals to obtain an integral transform of the sample reflectivity over the entire domain.

$$\frac{{I}_{D , Interference}}{{F}_{I}^{2} {C}_{1} \sqrt{{C}_{2}}} = {\int }_{- ∞}^{∞} a ( \vert τ \vert ) exp \{ i [ ω + 2 \frac{\partial {ϕ}_{T} ( τ )}{\partial τ} ] τ \} d τ . \qquad ( 11 )$$

The Fourier transform relationship then becomes apparent.

$$\begin{matrix}\hat{a} ( {f}_{eff} ) & = {\int }_{- ∞}^{∞} a ( \vert τ \vert ) exp \{ 2 π {if}_{eff} τ \} d τ \\ {f}_{eff} & = \frac{ω + 2 \frac{\partial {ϕ}_{T} ( τ )}{\partial τ}}{2 π} \\ {\hat{a}}_{S} ( {f}_{eff} ) & = \frac{{I}_{D , Interference}}{{F}_{I}^{2} {C}_{1} \sqrt{{C}_{2}}} ( {f}_{eff} )\end{matrix} \} \qquad ( 12 )$$

In this case, the conjugate variables are the time it takes for light to be reflected from different depths of the sample, and the effective frequency which is tuned via the delay line (or more precisely, each point of the effective frequency is mapped to tuning the delay line at a constant rate). Note that this is the simplest conceptual case of the effective frequency. The method can be simplified by using a chirp in the delay.

The total reflected field as a function of time (and ultimately depth) can be recovered by an inverse transform.

$$a ( \vert τ \vert ) = {\int }_{- ∞}^{∞} \hat{a} ( {f}_{eff} ) exp \{ - 2 π i {f}_{eff} τ \} {df}_{eff} \qquad ( 13 )$$

### Nyquist Sampling Constraints

The time integral in the continuous case derived above goes over all time, which is unphysical. In reality, the measurement limits will be truncated and the measurement points will be digital and discrete rather than continuous. This discreteness will impose Nyquist limits on the “bandwidth” (measurement depth) and measurement resolution. These may be determined in a manner directly comparable to the Nyquist theorem of signal processing. As the derivation is well known to those of skill in the art, only the main results are presented here.

The effective frequency interval Δf and sampling rate Δt are related as follows:

$$\begin{matrix}Δτ & = \frac{1}{N \cdot Δ {f}_{eff}} = \frac{π}{\frac{\partial {ϕ}_{T} ( τ )}{\partial τ} {\vert}_{max} - \frac{\partial {ϕ}_{T} ( τ )}{\partial τ} {\vert}_{min}} \\ Δ z & = \int \frac{c}{n ( τ )} d τ \approx \frac{c}{{n}_{avg}} Δτ = \frac{c}{{n}_{avg}} \cdot \frac{π}{\frac{\partial {ϕ}_{T} ( τ )}{\partial τ} {\vert}_{max} - \frac{\partial {ϕ}_{T} ( τ )}{\partial τ} {\vert}_{min}}\end{matrix} \} \qquad ( 14 )$$

Similarly, the maximum effective frequency translates into a sampling rate of:

$$\begin{matrix}{τ}_{\begin{matrix}measurement \\ time per point\end{matrix}} & \approx \frac{2 \cdot {z}_{max} \cdot {n}_{avg}}{c} \\ Δ {f}_{eff} & = \frac{1}{N \cdot Δτ} = \frac{1}{{τ}_{max}} = \frac{c}{2 \cdot {z}_{max} \cdot {n}_{avg}} \\ N & = \frac{1}{Δ {f}_{eff} \cdot Δτ} = \frac{2 \cdot {z}_{max} \cdot {n}_{avg}}{c \cdot π} [ \frac{\partial {ϕ}_{T} ( τ )}{\partial τ} {\vert}_{max} - \frac{\partial {ϕ}_{T} ( τ )}{\partial τ} {\vert}_{min} ] \\ {τ}_{\begin{matrix}measurement \\ time total\end{matrix}} & \approx {( \frac{2 \cdot {z}_{max} \cdot {n}_{avg}}{c} )}^{2} \frac{1}{π} [ \frac{\partial {ϕ}_{T} ( τ )}{\partial τ} {\vert}_{max} - \frac{\partial {ϕ}_{T} ( τ )}{\partial τ} {\vert}_{min} ] \\ {f}_{\begin{matrix}Maximum \\ Sampling \\ Rate\end{matrix}} & = \frac{N}{{τ}_{\begin{matrix}measurement \\ time total\end{matrix}}} = \frac{N}{N {τ}_{\begin{matrix}measurement \\ time per point\end{matrix}}} = \frac{1}{{τ}_{\begin{matrix}measurement \\ time per point\end{matrix}}} = \frac{c}{2 \cdot {z}_{max} \cdot {n}_{avg}}\end{matrix} \} \qquad ( 15 )$$

These constraints can be significant depending on the type of wave used in the device. Since the wave velocity appears in the expressions, there will be a major difference between a fast wave, e.g., light, versus a slow wave such as sound.

To provide a sample illustration, a thermo-optic phase shifter can be used. This device can be mapped to the effective frequency of the proposed OCT device and will help provide a sense of what performance can be expected and what applications are feasible.

The mathematical conventions used in the following discussion vary somewhat from those listed previously. Specifically, t=time, and T=temperature.

A basic thermo-optic phase shifter is a length of waveguide overlain with a heater, which is used to change the refractive index. The first order phase response of such a device is as follows:

$$\begin{matrix}{ϕ}_{T} ( t ) = β ( t ) \cdot L = n ( T ( t ) ) \cdot {β}_{0} \cdot L \\ \frac{\partial {ϕ}_{T} ( t )}{\partial t} = \frac{\partial n ( t )}{\partial T} \frac{\partial T}{\partial t} \cdot {β}_{0} \cdot L\end{matrix} \} \qquad ( 16 )$$

We can substitute this into the Nyquist conditions for the device to obtain analytical expressions for the resolution:

$$\begin{matrix}{Δτ}_{thermo - optic} & = \frac{π}{\frac{\partial n ( t )}{\partial T} \frac{\partial T}{\partial t} \cdot {β}_{0} \cdot L} \\ Δ {z}_{thermo - optic} & \approx \frac{c}{{n}_{avg}} \cdot \frac{π}{\frac{\partial n ( t )}{\partial T} \frac{\partial T}{\partial t} \cdot {β}_{0} \cdot L}\end{matrix} \} \qquad ( 17 )$$

Similarly, the per point measurement time and number of measurement points are:

$$\begin{matrix}{t}_{\begin{matrix}measurement \\ time per point\end{matrix}} & = {t}_{max} \approx \frac{2 \cdot {z}_{max} \cdot {n}_{avg}}{c} \\ N & = \frac{2 \cdot {z}_{max} \cdot {n}_{avg}}{c \cdot π} \frac{\partial n ( t )}{\partial T} \frac{\partial T}{\partial t} \cdot {β}_{0} \cdot L \\ {t}_{\begin{matrix}measurement \\ time total\end{matrix}} & = {( \frac{2 \cdot {z}_{max} \cdot {n}_{avg}}{c} )}^{2} \frac{1}{π} \frac{\partial n ( t )}{\partial T} \frac{\partial T}{\partial t} \cdot {β}_{0} \cdot L \\ {f}_{\begin{matrix}Maximum \\ Sampling \\ Rate\end{matrix}} & = \frac{c}{2 \cdot {z}_{max} \cdot {n}_{avg}}\end{matrix} \} \qquad ( 18 )$$

A particularly attractive feature of these results is that the resolution can be improved be increasing the length of the thermo-optic phase shifter.

There are a number of general intuitions that can be developed from these expressions:
The faster the time derivative of the phase change, the better the resolution.
The longer the phase shifter, the better the resolution.
Total measurement time is determined by the maximum depth you want to look into the sample.
The maximum sampling rate is determined by the maximum depth you want to look into the sample.
Slower waves will have longer measurement times and higher number of sampling points.
   Tables 2 and 3 below provide sample results, respectively, for a silicon waveguide phase shifter (SOI) (operating thermally) and a high speed fiber optic phase shifter (based on lithium niobate (LiNO₃).

TABLE 2
Silicon-on-Insulator (SOI) Waveguide

| | | | | | | Max. | Min. | |
|---|---|---|---|---|---|---|---|---|
| T-O | | Device | | Sample | # of | Sampling | Measurement | |
| Coefficient | λ | Length | Resolution | Depth | Pts. | Rate | Time Total | |
| 1.8 · 10⁻⁴ K⁻¹ | 1 μm | 1 mm | 83.276 m | 1 km | 24 | 0.15 MHz | 160 | μs |
| 1.8 · 10⁻⁴ K⁻¹ | 1 μm | 30 mm | 2.776 m | 1 km | 721 | 0.15 MHz | 0.005 | s |

TABLE 3
Fiber/LiNO₃ Waveguide

| | | | | | | | Minimum |
|---|---|---|---|---|---|---|---|
| | | | | | | Maximum | Measure- |
| Modulator | | | | Sample | # of | Sampling | ment Time |
| Speed | | Resolution | | Depth | Pts. | Rate | Total |
| 10 | GHz | 9.4 | cm | 1 km | 21,000 | 0.15 MHz | 0.142 s |
| 40 | GHz | 2.4 | cm | 1 km | 84,000 | 0.15 MHz | 0.567 s |
| 100 | GHz | 0.94 | cm | 1 km | 212,000 | 0.15 MHz | 1.417 s |

It is worth noting that in conventional signal processing, a known time signal is used and transformed to work with the frequency components. In this method, we do the reverse, namely start with a signal of modulated frequency and transform it to work with the time components. This has important differences that defy DSP intuition—rather than being concerned with minimum sampling rate as in conventional DSP, the present focuses on maximum sampling rate. This is easy to explain physically. For the interferogram to include points deep in the sample, we must measure long enough for light to travel to that point and back. The total measurement time is this round trip travel time multiplied by the number of points. This is the minimum measurement time. It is interesting because the alternative of measuring the time response would require extremely fast detectors and short integration times for short distances. Thus, the same advantages of the Fourier transform method are similar to that of spectroscopy.

Biomedical, geomorphic, remote sensing, et cetera. This invention can be used in all the same applications as conventional tomography. Additionally, the narrow spectral sensitivity can be useful for several new applications such as creating tomographic profiles of molecules if the operating wavelength is tuned to one of their absorption spectra peaks. The new method should also be able to be made more resistant to noise, which should extend the maximum range of the measurement.

### Example 1: Implementation of UPT

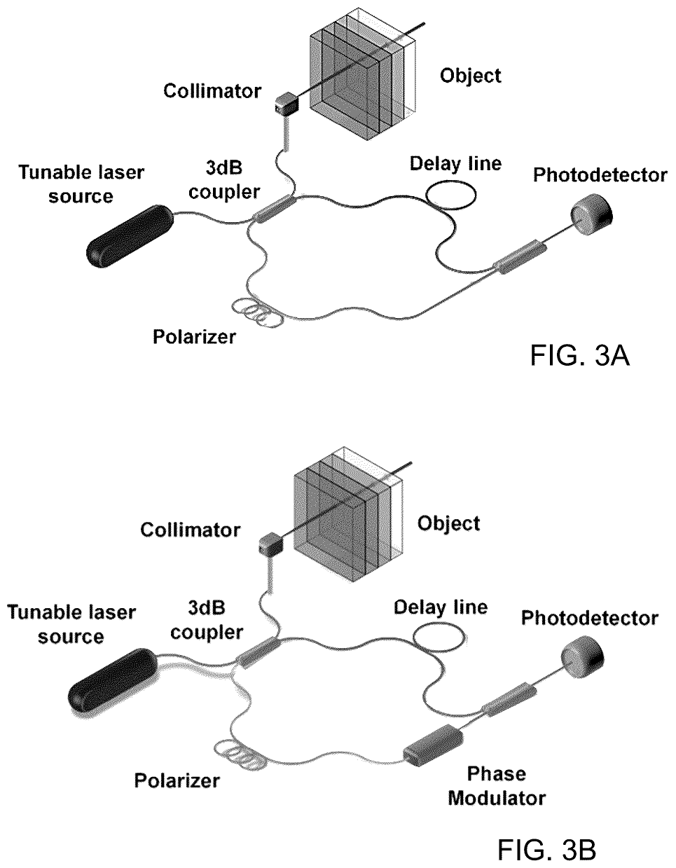

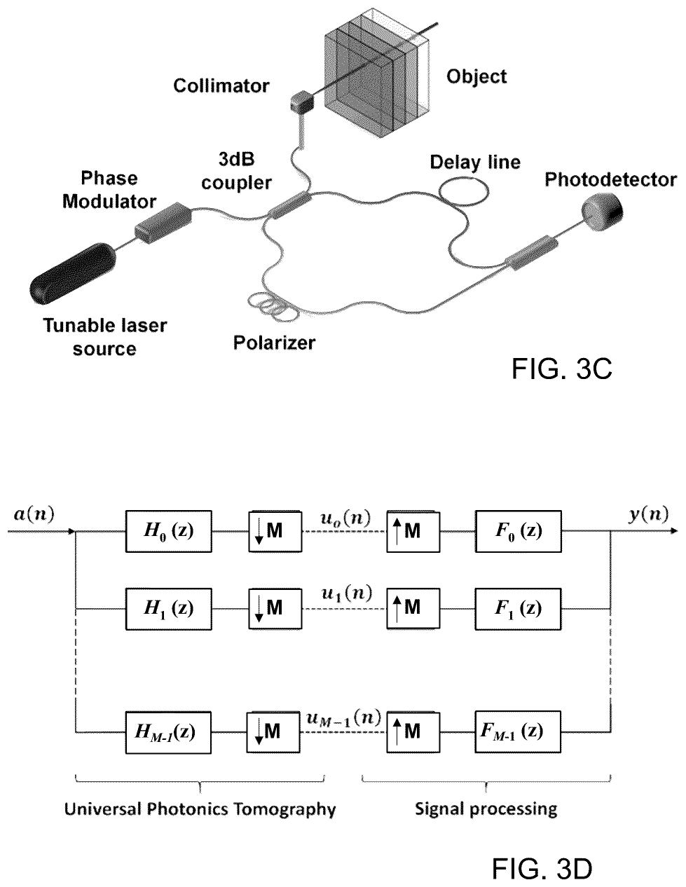

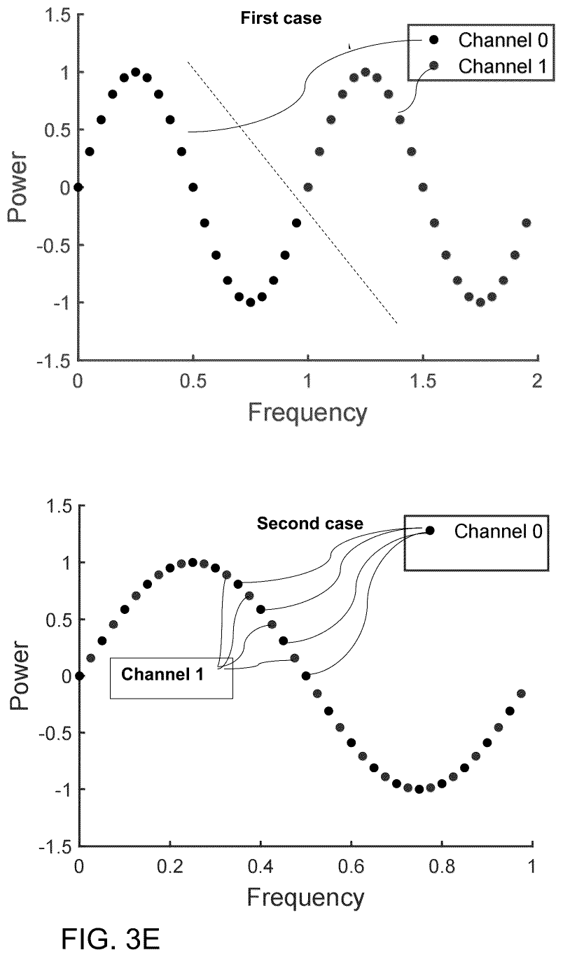

FIG. 3A illustrates a base case for an exemplary implementation of UPT without phase modulators, which resembles the Swept Source OCT in single mode fiber. The normalized interference term measured at the photodetector is given by:

$${P}_{intf} ( k ) = {\sum }_{i = - N}^{N} \overline{a} ( i ) exp ( \frac{j 2 π}{2 N + 1} ki ) \qquad ( 19 )$$

where ā (i) consists of reflection and transmission coefficients in the i-th surface present at a particular position with its magnitude determined from Fresnel equations and the transmitted optical power to the object, N is the total number of surfaces present, and each value of k represents a frequency in laser sweep. The negative arguments of the summation represent the conjugate part of the interference. ā(i) can be obtained by taking the Discrete Fourier Transform (DFT) of P_{intf}. The position, i, of the non-zero elements of ā(i) give the optical distance of the surface, while their magnitude can be used to determine the optical index of the layer which in turn can be used to extract the true physical distance.

Next, we add a phase modulator to the sample arm and use a signal generator to introduce a phase modulation ϕ(t). FIG. 3B shows a first variation on the base case in which a phase modulator is added in the sample arm. A waveform generator (not shown) is used to give slow modulation which assists in improving the resolution in length domain. Assuming that the modulation is slow compared to the time taken (time bin) by the laser to measure a single frequency, the DFT (transformation from k to n) of the interference term (P_{intf}) is given by,

$$2 N [ {P}_{intf} ( k ) ] ( n ) = {\vert F \vert}^{2} a ( n ) * h ( n ) + {\vert F \vert}^{2} a * ( - n ) * h * ( - n ) \qquad ( 20 )$$

where |F|²ā(n)=ā(i) for n>0 and |F|² is the transmitted optical power to the object. h(n)= [symbol] [exp(jϕ(kΔt))], where [symbol] [.] is the DFT function and Δt (time bin) is the time taken to measure the power at a single frequency. Eq. (20) can be truncated to n>0 regime and then normalized by |F|² to give u(n).

$$\\ \begin{matrix}u ( n ) = a ( n ) * h ( n ) & ( 21 )\end{matrix}$$

Eq. (21) resembles a filter h(n) applied to a(n) in a linear system with convolution in length (i.e., time) domain. A transfer function can then be defined in frequency domain, and this provides the opportunity to apply digital signal processing on the depth information.

FIG. 3C illustrates a second variation in which a phase modulator is added to the base case just after the tunable laser. A signal generator (not shown) is used to apply fast modulation which assists to increase the maximum unambiguous range. The fast modulation repeats after every sweep frequency, i.e., it is periodic with Δt. It can then be shown that the interference term is given by,

$$\\ \begin{matrix}{P}_{intf} ( k ) = {\sum }_{i = - N}^{N} \overline{a} ( i ) \overline{H} ( i ) exp ( \frac{j 2 π {υ}_{o} {l}_{o} ki}{c} ) & ( 22 )\end{matrix}$$

where H̅(i) is the autocorrelation function of the phase modulation. Eq. (22) can be written in the convolution form.

$$\\ \begin{matrix}u ( n ) = \tilde{a} ( n ) * h ( n ) & ( 23 )\end{matrix}$$

Here the P_{intf} has been replaced by u (n) and variable k is replaced n. ã(n)= [symbol] [ā(i)], and h(n)= [symbol] [H̅(i)] determines the filter coefficients. Note that here the convolution is in frequency domain, as opposed to previous case. Hence, the transfer function can be implemented in length domain.

Eq. 21 and Eq. 23 represent a linear system in which multirate signal processing can be used to increase the resolution of the system as shown in FIG. 3D, which provides a schematic for the working of UPT and the required post-processing in the filter bank form. The horizontal dashed lines indicate photodetection. The physical system of UPT corresponds to elements on the left-hand side (pre-photodetection process) of the filter bank scheme of FIG. 3D. The right-hand side (post-detection process), i.e., the “Signal Processing” elements are implemented digitally. The down-arrow and up-arrow blocks correspond to downsampling and upsampling respectively, both by a factor of an integer M. Upsampling is performed digitally, while downsampling is inbuilt in the UPT system as the resolution of the system is less than needed. The transfer functions (represented as Z transforms) are in frequency domain for the first case (FIG. 3B) while in length (i.e., time) domain for the second case (FIG. 3C). In FIG. 3D, uᵢ(n) represents the detected signal, Hᵢ(z) is the analysis filter, and Fᵢ(z) is the synthesis filter in the i^{th} channel. n is the time vector in the first case, and the frequency vector in the second case. Hᵢ(z) is implemented optically using a phase/intensity modulator while Fᵢ(z) is implemented digitally. a(n) is the high-resolution OCT information that we wish to obtain while y(n) is its reconstruction using the UPT system. Note that while Eq. (21) and Eq. (23) appear to be the same, the former is convolution in length domain while the latter in frequency domain. Hence, the interpretation of transfer function will be domain inverted compared to the previous case. Taking the Z-transform results in

$$\\ \begin{matrix}U ( z ) = A ( z ) H ( z ) & ( 24 )\end{matrix}$$

By performing multiple scans, axial resolution is improved in the first case (FIG. 3B) while maximum depth is increased in the second case (FIG. 3C). The first case may arise when bandwidth of laser is limited while second case may arise when frequency resolution is limited. These two cases are equivalent to the presence of a downsampled block in the system. Analysis filters are implemented using phase modulators while the synthesis filters and upsampling blocks are implemented digitally. In Example 2 below, we demonstrate a 2-channel filter bank for both the cases.

For slow modulation we use a linear phase modulation, which is effectively a z⁻¹ transfer function in Z domain. As the maximum bandwidth of the laser is usually limited, it may cause the resolution in length domain (axial resolution) to be less than desired, resulting in under sampling. Let the laser have a bandwidth that is M times smaller than required so that the axial resolution is down sampled by a factor of M from the desired lₒ. This can be depicted by a block diagram as shown in FIG. 3D. The block diagram resembles a single channel of M channel filter bank. If we make the measurement M times with M different synthesis filters (Hₘ), the ideally sampled signal can be reconstructed using analysis filters (Fₘ). On the other hand, Eq. (24) corresponds to a transfer function block with the Z-transform in length domain. The same multirate filter bank analysis can be used to deal with under sampling problem. In this case, downsampling is in frequency domain as the resolution of sweeping laser is limited. Hence, the filter bank can be used to reconstruct the signal with increased frequency resolution and detect object at greater depth without aliasing.

For demonstration purposes, we discuss the situation when M=2. The perfect reconstruction (PR) of a(n) is said to be achieved when y(n)=a(n−K), i.e., y(n) is perfect replica of a(n) and is with a shift of K points. This removes both aliasing and distortion from the reconstruction. For two channel filter bank, the PR condition is given by

$$\\ \begin{matrix}[ \begin{matrix}{F}_{0} ( z ) \\ {F}_{1} ( z )\end{matrix} ] = \frac{2 {z}^{{}_{} - L}}{Δ ( z )} [ \begin{matrix}{H}_{1} ( - z ) \\ - {H}_{0} ( - z )\end{matrix} ] & ( 15 )\end{matrix}$$

where Δ(z) is given by

$$\\ \begin{matrix}Δ ( z ) = {H}_{0} ( z ) {H}_{1} ( - z ) - {H}_{0} ( - z ) {H}_{1} ( z ) & ( 26 )\end{matrix}$$

The simplest implementation of this is the lazy filter bank, in which the first channel is detected without any modulation while the second channel shifts the input by one time

$$\\ \begin{matrix}{H}_{0} ( z ) = 1 ; {H}_{1} ( z ) = {z}^{{}_{} - 1} & ( 37 )\end{matrix}$$

$$\\ Δ ( z ) = - 2 {z}^{{}_{} - 1} ; K = 1$$

$$\\ {F}_{0} ( z ) = {z}^{{}_{} - 1} ; {F}_{1} ( z ) = 1$$

For the first case,

$$\\ z = exp ( \frac{- j 2 π k}{2 N + 1} ) .$$

Let total time of scan be T=NΔt. As only half of required number of points are scanned the phase modulation should be

$$\\ \begin{matrix}exp ( j ϕ ( t ) ) = exp ( \frac{- j π t}{T} ) & ( 48 )\end{matrix}$$

This corresponds to a linear phase modulation from 0 to π phase shift in time T and thus the voltage provided by signal generator vary from 0 to V_{π} in this time. For large N (of the order of 10,000), the assumption that ϕ(t) varies slowly in Δt holds.

For the second case, we assumed that the frequency of the modulation is comparable to c/lₒ, which can be of the order of 10s of megahertz. It is difficult as well as cost ineffective to produce arbitrary waveforms at such high frequency. The easiest modulation is sinusoidal, produced using an RF signal generator.

$$\\ \begin{matrix}exp ( j ϕ ( t ) ) = exp ( jA sin ( 2 π {f}_{m} t ) ) & ( 5 )\end{matrix}$$

fₘ is the sinusoidal phase modulation frequency and A is its amplitude. This gives

$$\\ \begin{matrix}\overline{H} ( i ) = {J}_{0} ( 2 A sin ( \frac{π {f}_{m} {il}_{o}}{c} ) ) & ( 60 )\end{matrix}$$

Where J₀ is the Bessel function of first kind and zeroth order. The corresponding filter coefficients and transfer function can be calculated from Eq. (30). To carry out the filter bank analysis, it is important that Δ(z) is invertible. For this purpose, the amplitude (A) and modulation frequency (fₘ) can be engineered so as to make the analysis filter stable. Alternatively, other types of waveforms can be used, but that would require high speed analog waveform generators.

For fast modulation, sinusoids are the only cost-effective option. The synthesis filters can be calculated from the perfect reconstruction conditions of filter banks, as given by Eq. 31 and Eq. 32, where K is an integer and corresponds to the delay due to signal processing.

$$\\ \begin{matrix}[ \begin{matrix}{F}_{0} ( z ) \\ {F}_{1} ( z )\end{matrix} ] = \frac{2 {z}^{{}_{} - K}}{Δ ( z )} [ \begin{matrix}{H}_{1} ( - z ) \\ - {H}_{0} ( - z )\end{matrix} ] & ( 31 )\end{matrix}$$

$$\\ \begin{matrix}Δ ( z ) = {H}_{0} ( z ) {H}_{1} ( - z ) - {H}_{0} ( - z ) {H}_{1} ( z ) & ( 32 )\end{matrix}$$

FIG. 3E illustrates samples of reconstruction signals formed by combining two channels (M=2) in the frequency domain for the first and second cases. The dots represent the effective frequency measured by both channels. In the first case (top), the results of both channels (the two channels are separated by the dashed line) contribute to extending the bandwidth in the frequency domain, thus improving time domain resolution. While in the second case (lower), the two channels interleave to increase the frequency resolution, thus extending the maximum ambiguous range. The graphs are presented to give an intuition of the placement of frequency points in the reconstructed signal and do not represent a physical situation.

### Example 2: UPT Results

The following results demonstrate the working principal of the inventive UPT approach under the universal framework. We then experimentally demonstrate how various modulation schemes provides the opportunity for novel detection and post-processing strategies.

The laser used for performing all the experiments was the 81608A Tunable Laser Source from Keysight Technologies (Santa Rosa, CA, US) which can give frequency resolution up to 0.1 μm and has a narrow linewidth (<10 kHz). The photodetector is the 81635A Dual Optical Power Sensor, also from Keysight. The phase modulator employed in both the cases is the Thorlabs Lithium Niobate 40 GHz phase modulators (LN27S-FC). The linear waveform is produced using Keysight B2960 series power supply while the sinusoidal signal is generated using Keysight MXG series 6 GHz Analog Signal Generator. The entire setup (excluding objects) is built upon SMF-28 single mode fiber.

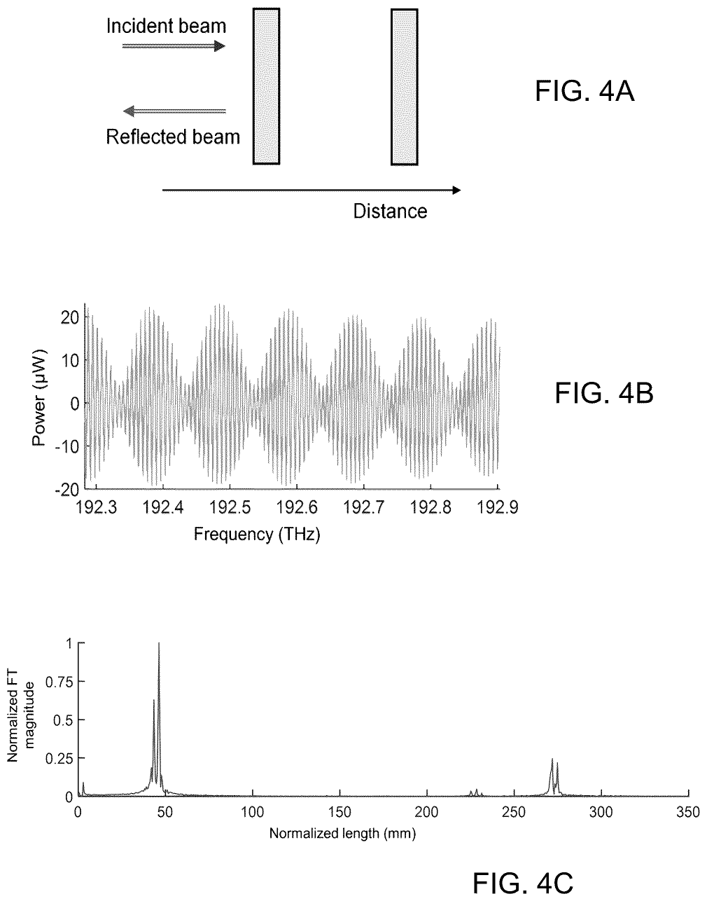

To demonstrate how to increase the axial resolution using the inventive UPT, we used two microscope slides as objects (FIG. 4A), one placed directly in front of the other, hence a total of four different interface surfaces separating two different media (namely air and glass). The microscope slides are about 1 mm thick, and the two slides are placed about 12 cm apart. The refractive index of glass is assumed to be n_{glass}≈1.5, and the refractive index of air is taken to be nₐᵢᵣ≈1.0. This creates a situation where the bandwidth of laser is not high enough to clearly distinguish the two surfaces of the slide. The laser sweeps a bandwidth of 1 nm with 0.2 pm resolution. This results in an axial resolution (lₒ) of 2.4 mm, while the normalized distance between the slide surfaces is 3 mm.

Base case: To establish the base case without modulators, the measured interference pattern on the photodetector as a function of frequency sweep is shown in FIG. 4B after using an offset equal to its mean. The bandwidth is 5 nm at a wavelength of 1.55 μm and resolution (υo) is 0.3 pm. FIG. 4C provides the Fourier transform (FT) of the interference pattern. The four larger peaks clearly distinguish the four surfaces and predict the distances between them. The smaller peaks (barely visible) are due to autocorrelation of the sample arm signal in the interferogram and can be removed by balanced photodetection.

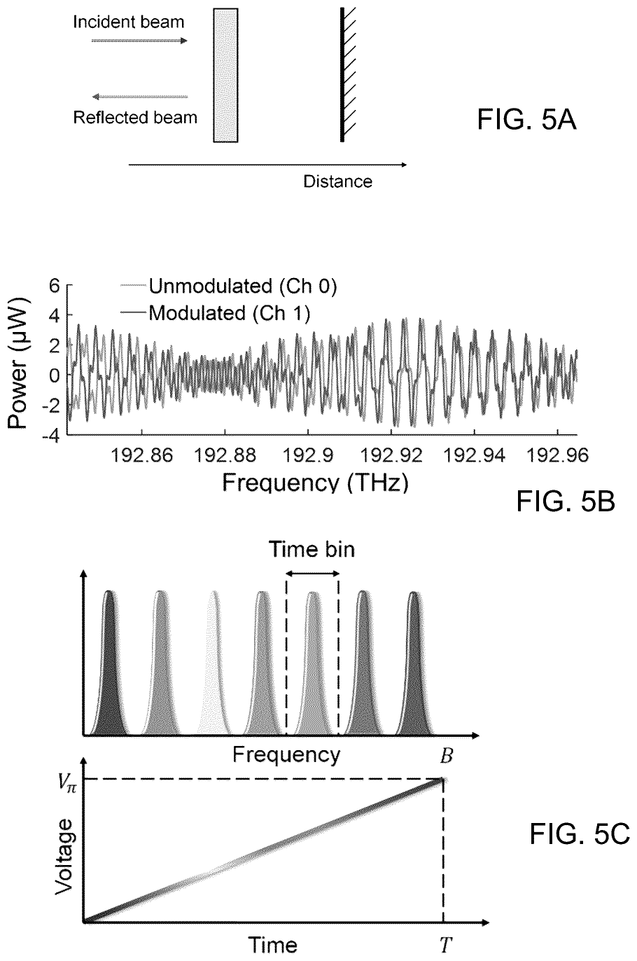

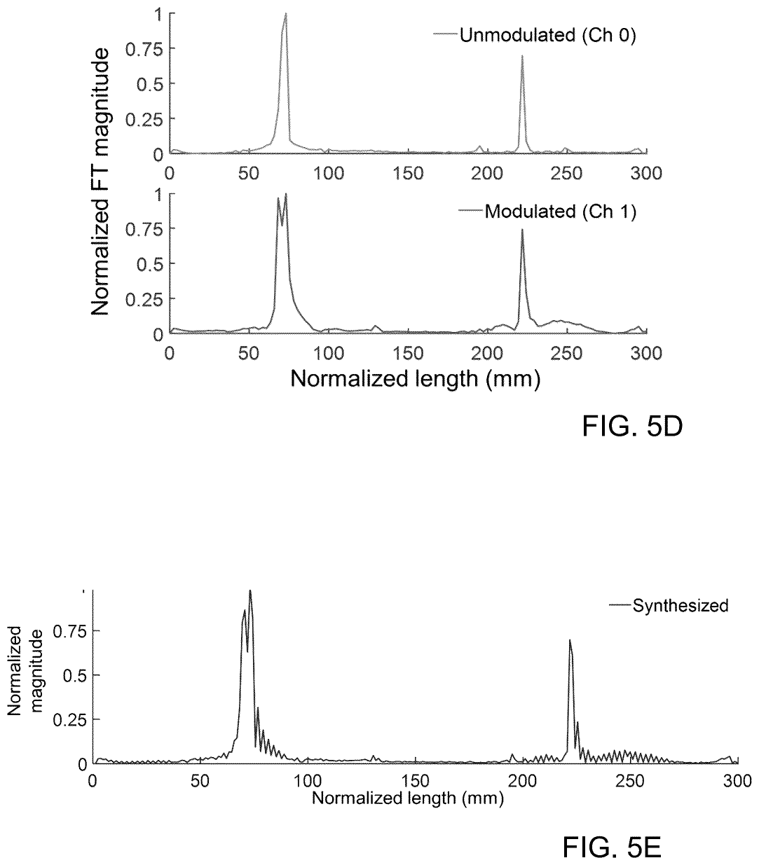

Increasing axial resolution: To demonstrate how to increase the axial resolution, we use a microscope slide and a mirror behind it, as shown in FIG. 5A. We create a situation where the bandwidth of laser is not high enough to clearly distinguish the two surfaces of the slide. The laser sweeps a bandwidth of 1 nm with 0.2 pm resolution. This results in an axial resolution (lₒ) of 2.4 mm, while the normalized distance between the slide surfaces is 3 mm. This measurement referred to as unmodulated signal, (curve Ch 0 in FIGS. 5B and 5D) corresponds to conventional SS-OCT, but the surfaces are barely resolvable due to limited bandwidth of the tunable laser source. Next, we use a waveform generator to provide a linear phase modulation to the sample arm, as shown in FIG. 5C. The interferogram that is obtained can distinguish the surfaces better or worse depending on the position of the surfaces, but the resolution (lₒ) remains the same (FIG. 5D, lower curve). The two signals are combined and treated as two different channels of a multirate filter bank (FIG. 5E). This improves lₒ from 2.4 mm to 1.2 mm. The surfaces can be distinguished more easily now, and their positions are known twice as accurately as before. Thus, the axial resolution of the synthesized signal with a 1 nm bandwidth optical source is equal to that of a single channel system with a 2 nm source—a 100% improvement. Further, note that multiple channels can be used to improve the axial resolution even more. This is a highly significant result, as it provides the best path to ultrahigh resolution devices by a large margin.

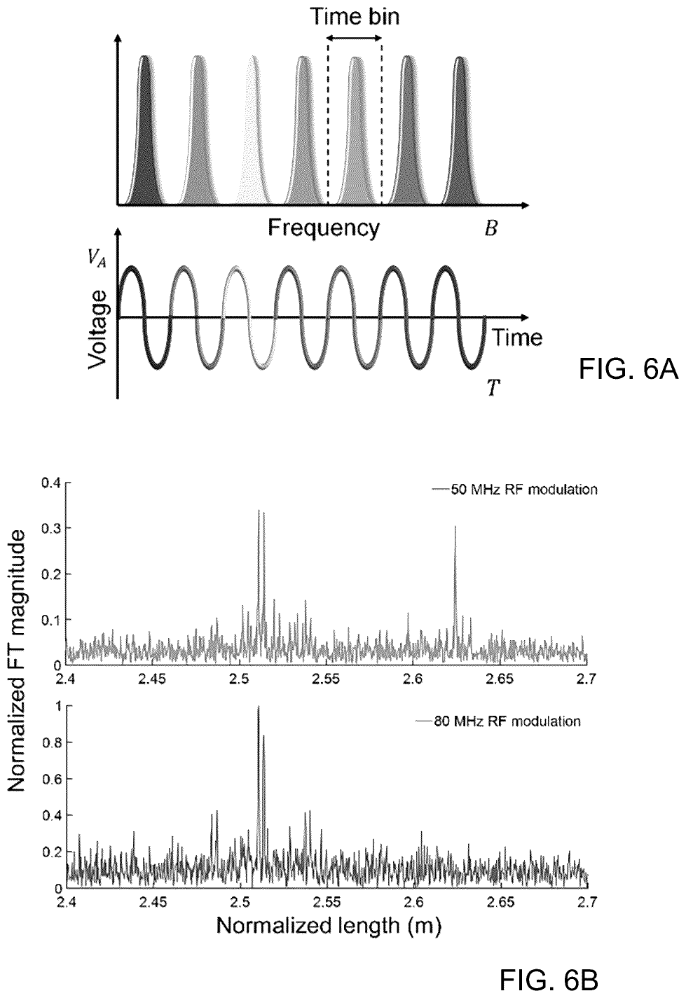

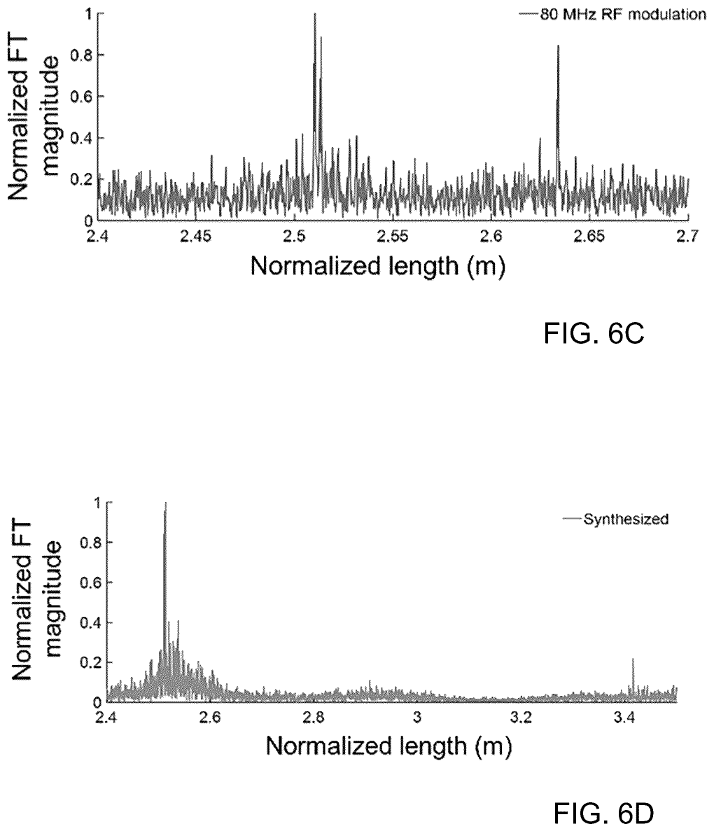

Increasing Maximum Depth: For a simple demonstration on how to increase the maximum unambiguous depth, we again use the microscope slide with a mirror behind it (see FIG. 5A). We define a balanced point which is the zero position in the length domain and physically represents the point where delay of reference signal is equal to that of signal from the object. The microscope slide is used as a reference, which is at 2.51 m from the balanced point, while the mirror, which is at 3.41 m from the balanced point, is the target object. FIG. 6A provides a schematic depicting the voltage applied to phase modulation as laser frequency is tuned. Here we consider the situation when the resolution of laser sweep is limited to 0.4 pm, which corresponds to maximum unambiguous depth (L) equal to 3 m, and the position of the target (mirror) is beyond it. We first measure this object with 50 MHz sinusoidal phase modulation as shown in FIG. 6B, upper curve. The peak for mirror appears at 2.62 m which is an aliasing artifact that arises due to undersampled measurement. To predict the true position of the target we perform a second measurement where the transfer function of the phase modulation has a zero at the unaliased position of the target but not at the aliased position. If the target peak disappears then it indicates that the target is indeed at much further distance, otherwise the original peak gives the correct position. Thus, we use adaptive phase modulation and signal processing to determine the position of a single target which is often the requirement of a conventional LiDAR system. This is a valuable method as it is often difficult to determine the accurate transfer function of the optical modulation due to nonlinearity, variable V_{π}, RF impedance mismatch, etc., but this method only requires the knowledge of zero crossings of the transfer function. In our case, an 80 MHz sinusoidal phase modulation gives a transfer function that has a zero at 3.41 m and we show that this makes the 2.62 m peak disappear (FIG. 6B, lower curve). Therefore, we can conclude that position of the target is actually at 3.41 m. We also demonstrate in FIG. 6C that the peak would not have disappeared if the true position of the mirror were actually at 2.62 m, by physically placing a mirror at this position. Also, the 50 MHz and 80 MHz measurements can be treated as two different channels in a multirate filter bank and combined, as shown in FIG. 6D, to give a graph that has twice the maximum unambiguous range than individual channels. This method will perform better for more complex objects but also requires an accurate structure of the analysis of the transfer function produced by phase modulation. This demonstrates that distances up to 6 m can be measured by using laser sweep resolution which corresponds to only maximum depth of 3 m in the unmodulated case. As mentioned above, multiple channels (scans) can be used to increase the limit even more. Also, for simple targets, adaptive measurements can be performed which will require lesser number of channels and can still measure more distant positions of the target. This is a highly significant result for the same reasons.

The above discussion demonstrates UPT as a universal method to measure depth and position of objects at various distances by adjusting the laser sweep frequency and bandwidth. The inventive UPT framework provides an alternative approach to improve the resolution and/or depth performance through the use of slow and/or fast modulation of the optical carrier. This approach requires only a simple phase modulator and waveform/signal generator which are more economical and easier to integrate in the system. By making multiple scans, ultrahigh resolutions can be achieved both in frequency and length domain. The only drawback in this method is the extra time required to perform multiple scans. The design is agnostic to the type of phase modulators used, which can be mechanical, acousto-optic, electro-optic, etc. In our experiments, we used Lithium Niobate phase modulators which have promising specifications of low V_{π} and high RF bandwidths.

This multichannel detection scheme works on the principle of multirate filter banks, and the number of channels can be increased to more than two and can be used for more complex objects, similar to how a multichannel filter bank works. Given enough channels with appropriate modulation, they can be theoretically combined by multirate signal processing to get a reconstructed signal with arbitrarily high resolution. In the multirate filter bank formulation, the resolution improvement has no theoretical limit. However, physically speaking, for long distances, the detected power might drop below the noise levels of the photodetectors. Another practical challenge that exists is the imprecision in the frequency sweep. If all the frequency values reported by the laser do not have constant frequency difference, the Fourier transform will be noisy when making a measurement near or beyond the Nyquist limit. We observe this in the second case where the noise floor is due to the improperly spaced frequency values. The power on the photodetector comprises of the DC term (reference autocorrelation), the sample autocorrelation and the interference term (cross-correlation). To efficiently extract the interference term with high SNR, it is important to filter out the remaining two terms. One way is to attenuate the signal in the sample arm and subtract the mean of the total interference power. This method can still produce small peaks in the Fourier transform due to presence of autocorrelation term, which can also be observed in the base case (FIGS. 4A-4C). A better way to remove the other two terms would be to use balanced photodetection, where subtracting the two interference powers cancels out the two unnecessary terms.

To implement synthesis filters, it is essential that Δ(z) as described in Eq. (32) is invertible. This is not the case when sinusoidal phase modulation is given to only one channel with no modulation on the other. Thus, for second case, both channels should have sinusoidal modulation. Other modulation shapes can also be used if the speed of waveform generator permits. Under the UPT framework other novel configurations are also possible, for example, using intensity modulators instead of phase modulators to implement more complex filters, or developing the system similar to SD-OCT and using optical modulation to virtually improve the bandwidth of the source and frequency resolution of the spectrum analyzer.

From an engineering standpoint the most significant results are the improvements in axial resolution and maximum depth measurement without increasing the signal bandwidth and frequency resolution of tunable laser. This is because many factors form a hard limit on the source bandwidth in conventional systems. Specifically, these include source limitations, transparency windows of the optical components, and power tolerance. Similarly, frequency resolution is limited by factors depending on the tunable laser, for example, external cavity lasers require large cavities for small free spectral range. Operation under the UPT framework bypasses all these hardware challenges without the need for exotic and costly equipment.

### Method for Optimizing Adiabatic Tapers

A review of the well-known results of basic coupled mode theory is the starting point for this disclosure. Here, the waveguides are assumed to be composed of nonmagnetic and dielectric materials, which simplifies the mathematics while still illustrating the fundamental concepts underlying the method. The most general form of the derivation that relaxes these conditions is found using the approach of Koegelnik's well-known coupled wave theory (See, e.g., H. Koegelnik, “Theory of Dielectric Waveguides,” in Integrated Optics Topics in Applied Physics, vol. VII, T. Tamir, Ed., Berlin, Heidelberg, Springer, 1975, pp. 66-79, which is incorporated herein by reference.)

In this formalism, the electric and magnetic fields are expressed as a combination of eigenmodes that are determined by Maxwell's equations combined with the boundary conditions imposed by the waveguide geometry and composition. The effect of a perturbation to the waveguide permittivity is to transfer energy from one mode to another.

For single frequency fields:

$$\\ \begin{matrix}∇ \cdot ( {E}^{{}_{} *} \times {H}^{{}_{} ′} ) = {H}^{{}_{} ′} \cdot ∇ \times {E}^{{}_{} *} - {E}^{{}_{} *} \cdot ∇ \times {H}^{{}_{} ′} = {H}^{{}_{} ′} \cdot i ω μ {H}^{{}_{} *} - {E}^{{}_{} *} \cdot i ω {ε}^{{}_{} ′} {E}^{{}_{} ′} - {E}^{{}_{} *} \cdot {J}^{{}_{} ′} & ( 33 )\end{matrix}$$

$$\\ ∇ \cdot ( {E}^{{}_{} ′} \times {H}^{{}_{} *} ) = {H}^{{}_{} *} \cdot ∇ \times {E}^{{}_{} ′} - {E}^{{}_{} ′} \cdot ∇ \times {H}^{{}_{} *} = {H}^{{}_{} *} \cdot - i ω {μ}^{{}_{} ′} {H}^{{}_{} ′} - {E}^{{}_{} ′} \cdot - i ω {εE}^{{}_{} *} - {E}^{{}_{} ′} \cdot {J}^{{}_{} *}$$

Add the two equations to obtain the basis for the coupled mode relationship.

$$\\ \begin{matrix}∇ \cdot ( {E}^{{}_{} *} \times {H}^{{}_{} ′} ) + ∇ \cdot ( {E}^{{}_{} ′} \times {H}^{{}_{} *} ) = - i ω ( {ε}^{{}_{} ′} - ε ) {E}^{{}_{} *} \cdot {E}^{{}_{} ′} - i ω ( {μ}^{{}_{} ′} - μ ) {H}^{{}_{} ′} \cdot {H}^{{}_{} *} - {E}^{{}_{} *} \cdot {J}^{{}_{} ′} - {E}^{{}_{} ′} \cdot {J}^{{}_{} *} = - i ω Δ ε {E}^{{}_{} *} \cdot {E}^{{}_{} ′} - i ω Δ μ {H}^{{}_{} ′} \cdot {H}^{{}_{} *} - {E}^{{}_{} *} \cdot {J}^{{}_{} ′} - {E}^{{}_{} ′} \cdot {J}^{{}_{} *} & ( 34 )\end{matrix}$$

Integrate over the volume of space and Apply Gauss' theorem to the left side:

$$\\ \begin{matrix}{\int \int }_{\partial S} [ ∇ \cdot ( {E}^{{}_{} *} \times {H}^{{}_{} ′} ) + ∇ \cdot ( {E}^{{}_{} ′} \times {H}^{{}_{} *} ) ] \cdot dV = {\int \int }_{\partial S} ( {E}^{{}_{} *} \times {H}^{{}_{} ′} + {E}^{{}_{} ′} \times {H}^{{}_{} *} ) \cdot dS = - i ω {\int \int \int }_{V} Δ ε {E}^{{}_{} *} \cdot {E}^{{}_{} ′} dV - i ω {\int \int \int }_{V} Δ μ {H}^{{}_{} ′} \cdot {H}^{{}_{} *} dV - {\int \int \int }_{V} ( {E}^{{}_{} *} \cdot {J}^{{}_{} ′} + {E}^{{}_{} ′} \cdot {J}^{{}_{} *} ) dV & ( 35 )\end{matrix}$$

Consider the limit in which the transverse integral is taken infinitely far away in the plane perpendicular to propagation, and that the integral in the direction of propagation is infinitesimally small. Taking z as the propagation direction, since physical fields vanish at infinity the integral reduces to:

$${lim}_{Δ z \to 0} \{ \begin{matrix}{\int \int }_{S} [ ( {E}^{*} \times {H}^{′} + {E}^{′} \times {H}^{*} ) {\vert}_{z + Δ z} - ( {E}^{*} \times {H}^{′} + {E}^{′} \times {H}^{*} ) {\vert}_{z} ] \cdot {n}_{z} = \\ - i ω Δ z {\int \int }_{S} Δε {E}^{*} \cdot {E}^{′} dSS - i ωΔ z {\int \int \int }_{V} Δ μ {H}^{′} \cdot {H}^{*} dV - \\ Δ z {\int \int \int }_{V} ( {E}^{*} \cdot {J}^{′} + {E}^{′} \cdot {J}^{*} ) dV\end{matrix} \} \qquad ( 36 )$$

Next, apply the fundamental theorem of calculus to write how the fields vary with one another in the propagation direction:

$${\int \int }_{S} \frac{d}{dz} ( {E}^{*} \times {H}^{′} + {E}^{′} \times {H}^{*} ) \cdot {n}_{z} dS = - i ω {\int \int }_{S} Δε {E}^{*} \cdot {E}^{′} dS - i ω {\int \int }_{S} Δμ {H}^{′} \cdot {H}^{*} dS - {\int \int }_{S} ( {E}^{*} \cdot {J}^{′} + {E}^{′} \cdot {J}^{*} ) dS \qquad ( 37 )$$

Next, expand the fields in terms of the mode expansion of the unperturbed system. Note that the perturbed fields will have variable amplitude coefficients since they are not in their natural basis.

$$E ( x , y , z , t ) = {A}_{l} {E}_{l} ( x , y ) exp [ i ( ω t - {β}_{l} z + {φ}_{l} ) ] \\ H ( x , y , z , t ) = {A}_{l} {H}_{l} ( x , y ) exp [ i ( ω t - {β}_{l} z + {φ}_{l} ) ] \\ {E}^{′} ( x , y , z , t ) = {A}_{m} ( z ) {E}_{m} ( x , y ) exp [ i ( ω t - {β}_{m} z + {φ}_{m} ) ] \\ {H}^{′} ( x , y , z , t ) = {A}_{m} ( z ) {H}_{m} ( x , y ) exp [ i ( ω t - {β}_{m} z + {φ}_{m} ) ] \qquad ( 38 )$$

The permittivity perturbation causes coupling between a waveguide modes. To see this, using the above mode convention, substitute the following fields into the field coupling equation. For the unprimed field we take one incident waveguide mode, and for the primed field take the unknown projection of that field in the perturbed system. Similarly, use Ohm's law to express the free currents in terms of the fields and conductivity.

$${\int \int }_{S} \frac{d}{dz} ( {E}^{*} \times {H}^{′} + {E}^{′} \times {H}^{*} ) \cdot {n}_{z} dS = - i ω {\int \int }_{S} Δε {E}^{*} \cdot {E}^{′} dS - i ω {\int \int }_{S} Δμ {H}^{′} \cdot {H}^{*} dS - {\int \int }_{S} ( {E}^{*} \cdot {J}^{′} + {E}^{′} \cdot {J}^{*} ) dS \to \frac{d}{dz} \{ {A}_{l} {A}_{m} ( z ) exp [ i ( {β}_{l} - {β}_{m} ) z + i ( {φ}_{m} - {φ}_{l} ) ] \} {\int \int }_{S} [ {E}_{l} ( x , y ) \times {H}_{m} ( x , y ) + {E}_{m} ( x , y ) \times {H}_{l} ( x , y ) ] \cdot {n}_{z} dS = - i ω {\int \int }_{S} Δε ( x , y , z ) {A}_{l} {A}_{m} ( z ) exp [ i ( {β}_{l} - {β}_{m} ) z + i ( {φ}_{m} - {φ}_{l} ) ] {E}_{l} ( x , y ) \cdot {E}_{m} ( x , y ) dS - i ω {\int \int }_{S} Δμ ( x , y , z ) {A}_{l} {A}_{m} ( z ) exp [ i ( {β}_{l} - {β}_{m} ) z + i ( {φ}_{m} - {φ}_{l} ) ] {H}_{l} ( x , y ) \cdot {H}_{m} ( x , y ) dS - {\int \int }_{S} ( {σ}^{′} + σ ) {A}_{l} {A}_{m} ( z ) exp [ i ( {β}_{l} - {β}_{m} ) z + i ( {φ}_{m} - {φ}_{l} ) ] {E}_{l} ( x , y ) \cdot {E}_{m} ( x , y ) dS \qquad ( 39 )$$

Note that may be a technical issue concerning the behavior of the z-components of the fields arising from the orthogonality condition that requires the perturbations to be small for this expression to be accurate. This can be trivially satisfied as adiabatic tapers are inherently gradual. In other contexts, however, large perturbations can be handled by a slight modification to the portion of the coupling coefficient arising from the longitudinal fields.

Next, use the orthogonality condition to simplify the left hand side:

$$\frac{d}{dz} [ 2 {A}_{l} {A}_{l} ( z ) ] {\int \int }_{S} [ {E}_{l} ( x , y ) \times {H}_{l} ( x , y ) ] \cdot {n}_{z} dS = - i ω {\int \int }_{S} Δε ( x , y , z ) {A}_{l} {A}_{m} ( z ) exp ( i {Δβ}_{lm} z + i Δ {φ}_{ml} ) {E}_{l} ( x , y ) \cdot {E}_{m} ( x , y ) dS - i ω {\int \int }_{S} Δμ ( x , y , z ) {A}_{l} {A}_{m} ( z ) exp ( i {Δβ}_{lm} z + i {Δφ}_{ml} ) {H}_{l} ( x , y ) \cdot {H}_{m} ( x , y ) dS - {\int \int }_{S} ( {σ}^{′} + σ ) {A}_{l} {A}_{m} ( z ) exp ( i {Δβ}_{lm} z + i {Δφ}_{ml} ) {E}_{l} ( x , y ) \cdot {E}_{m} ( x , y ) dS \\ {Δβ}_{lm} = {β}_{l} - {β}_{m} \\ {Δφ}_{ml} = {φ}_{m} - {φ}_{l} \qquad ( 40 )$$

Isolate the amplitude and simplify:

$$\frac{{dA}_{l} ( z )}{dz} = \qquad ( 41 )$$

$$- i \frac{ω}{2} {A}_{m} ( z ) exp ( i {Δβ}_{lm} z + i {Δφ}_{ml} ) \frac{{\int \int }_{S} Δε ( x , y , z ) {E}_{l} ( x , y ) \cdot {E}_{m} ( x , y ) dS}{{\int \int }_{S} [ {E}_{l} ( x , y ) \times {H}_{l} ( x , y ) ] \cdot {n}_{z} dS} -$$

$$i \frac{ω}{2} {A}_{m} ( z ) exp ( i {Δβ}_{lm} z + i {Δφ}_{ml} ) \frac{{\int \int }_{S} Δμ ( x , y , z ) {H}_{l} ( x , y ) \cdot {H}_{m} ( x , y ) dS}{{\int \int }_{S} [ {E}_{l} ( x , y ) \times {H}_{l} ( x , y ) ] \cdot {n}_{z} dS} -$$

$$\frac{1}{2} {A}_{m} ( z ) exp ( i {Δβ}_{lm} z + i {Δφ}_{ml} ) \frac{{\int \int }_{S} ( {σ}^{′} + σ ) {E}_{l} ( x , y ) \cdot {E}_{m} ( x , y ) dS}{{\int \int }_{S} [ {E}_{l} ( x , y ) \times {H}_{l} ( x , y ) ] \cdot {n}_{z} dS}$$

Finally, express in terms of coupling coefficients:

$$\frac{{dA}_{l} ( z )}{dz} = ( {κ}_{lm ε} + {κ}_{lm μ} + {κ}_{lm σ} ) {A}_{m} ( z ) exp ( i Δ {β}_{lm} z + i Δ {φ}_{ml} ) \\ {κ}_{lm ε} = - i \frac{ω}{2} \frac{{\int \int }_{S} Δε ( x , y , z ) {E}_{l} ( x , y ) \cdot {E}_{m} ( x , y ) dS}{{\int \int }_{S} [ {E}_{l} ( x , y ) \times {H}_{l} ( x , y ] \cdot {n}_{z} dS} \\ {κ}_{lm μ} = - i \frac{ω}{2} \frac{{\int \int }_{S} Δμ ( x , y , z ) {H}_{l} ( x , y ) \cdot {H}_{m} ( x , y ) dS}{{\int \int }_{S} [ {E}_{l} ( x , y ) \times {H}_{l} ( x , y ) ] \cdot {n}_{z} dS} \\ {κ}_{lm σ} = - \frac{1}{2} \frac{{\int \int }_{S} ( {σ}^{′} + σ ) {E}_{l} ( x , y ) \cdot {E}_{m} ( x , y ) dS}{{\int \int }_{S} [ {E}_{l} ( x , y ) \times {H}_{l} ( x , y ) ] \cdot {n}_{z} dS} \qquad ( 42 )$$

By inspection, the form of the most general coupling equation is suitable for application of the optimization method described below.

The fields may be decomposed in terms of the mode amplitudes Ar.

$$E = {\sum }_{l} {A}_{l} {E}_{l} ( x , y ) exp [ i ( ω t - {β}_{l} z + {φ}_{l} ) ] \qquad ( 43 )$$

The mode orthonormalization is chosen so that the modal fields carry a power P:

$$\frac{1}{2} {\int }_{∞}^{∞} {\int }_{∞}^{∞} {E}_{l} ( x , y ) \times {H}_{m} ( x , y ) dxdy = P {δ}_{lm} \qquad ( 44 )$$

The differential equations that govern the mode amplitudes are:

$$\frac{{dA}_{l} ( z )}{dz} = - {κ}_{lm} {A}_{m} ( z ) exp [ i ( {β}_{l} - {β}_{m} ) z + i ( {φ}_{l} - {φ}_{m} ) ] \\ \frac{{dA}_{m} ( z )}{dz} = ∓ {κ}_{ml} {A}_{l} ( z ) exp [ - i ( {β}_{l} - {β}_{m} ) z - i ( {φ}_{l} - {φ}_{m} ) ] \\ {κ}_{lm} = \frac{ω}{4 \cdot P} {\int }_{∞}^{∞} {\int }_{∞}^{∞} {E}_{l} ( x , y ) \cdot Δε ( x , y ) {H}_{m} ( x , y ) dxdy \qquad ( 45 )$$

The strength of the coupling is governed by coefficient k, which is a function of the dielectric perturbation and the extent to which it overlaps with the interacting modes. The sign difference between the equation depends on whether the modes are co-propagating or counter-propagating.

Essentially, the inventive method devises an adiabatic taper by formally minimizing the mode amplitudes along the taper. Since all possible mode interactions are governed by this equation, this will result in a taper that is optimized in the most general sense.

In a tapered waveguide, the strength of the coupling coefficient will become variable along the taper, as well as the mode properties. Notably, the variation of the propagation constant is equal to the self-coupling coefficient (e.g. Δβ₁=kₗₗ). Following Eq. (45), for a z-dependent taper the mode amplitudes may be expressed as the integrals of the form:

$${A}_{l} ( z ) = \int - {κ}_{lm} ( z ) {A}_{m} ( z ) exp \{ i [ {β}_{l} ( z ) - {β}_{m} ( z ) ] z + i ( {φ}_{l} - {φ}_{m} ) \} dz \qquad ( 76 )$$

Minimizing the amplitudes results in a condition of the form:

$$0 = {dA}_{l} ( z ) = [ \begin{matrix}\int - \frac{d {κ}_{lm} ( z )}{dz} {A}_{m} ( z ) exp \{ i [ {β}_{l} ( z ) - {β}_{m} ( z ) ] z + \\ i ( {φ}_{l} - {φ}_{m} ) \} dz - \\ \int {κ}_{lm} ( z ) exp \{ i [ {β}_{l} ( z ) - {β}_{m} ( z ) ] z + i ( {φ}_{l} - {φ}_{m} ) \} {dA}_{m} ( z ) - \\ \int {κ}_{lm} ( z ) {A}_{m} ( z ) i \{ \begin{matrix}z [ \frac{d {β}_{l} ( z )}{dz} - \frac{d {β}_{m} ( z )}{dz} ] + \\ [ {β}_{l} ( z ) - {β}_{m} ( z ) ]\end{matrix} \} \\ exp \{ i [ {β}_{l} ( z ) - {β}_{m} ( z ) ] z + i ( {φ}_{l} - {φ}_{m} ) \} dz\end{matrix} ] \qquad ( 47 )$$

This second term vanishes identically from the condition that dA=0. Otherwise the remaining terms must vanish to meet the adiabatic condition. Applying this condition to the coupling coefficient will enable us to determine the optimal permittivity taper. The solution of this equation is straightforward, however in the most general case it will need to be performed numerically (because the propagation constants generally can't be solved analytically).

The variation of the coupling coefficient throughout a taper occurs not only through dielectric perturbation, but also through the evolution of the normal mode field profiles (represented below as the correction terms ΔE₁ and ΔHₘ). For a z-dependent taper the coupling coefficient may be expressed as:

$${κ ( z )}_{lm} = \qquad ( 48 )$$

$$\frac{ω}{4 \cdot P} {\int }_{∞}^{∞} {\int }_{∞}^{∞} [ {E}_{l} ( x , y ) + Δ {E}_{l} ( x , y , z ) ] \cdot Δε ( x , y , z ) [ {H}_{m} ( x , y ) + Δ {H}_{m} ( x , y , z ) ] dxdy$$

If we consider only a small step in the taper, then all the correction terms and the perturbation will be small. This results in a first order expression for the coupling coefficient as:

$${κ ( z )}_{lm} \approx \frac{ω}{4 \cdot P} {\int }_{∞}^{∞} {\int }_{∞}^{∞} {E}_{l} ( x , y ) \cdot Δε ( x , y , z ) {H}_{m} ( x , y ) dxdy = \frac{ω}{4 \cdot P} {\int }_{∞}^{∞} {\int }_{∞}^{∞} {E}_{l} ( x , y ) \cdot \frac{Δε ( x , y , z )}{Δ z} Δ {zH}_{m} ( x , y ) dxdy \qquad ( 49 )$$

Adiabatic transitions will be gradual, so the first order approximation will be valid for the structures under consideration. This expression is perfectly amenable to the type of optimization we wish to perform.

This solves the problem for the case of a continuously variable perturbation, however it is straightforward to extend to all the other important cases (such as waveguide bending or width tapering) using the technique of conformal mapping.

The technique of conformal mapping reformulates a waveguide that is curved within the x-z plane into an equivalent straight waveguide in the conformal u-v plane (along lines of u=constant). The new mapping is compatible with standard numerical mode solvers, although it comes at a price of complicating the refractive index profile in the plane transverse to propagation. The problem of converting a graded index change to an equivalent taper profile may be arrived at in a manner similar to the derivation of M. Heiblum and J. H. Harris in “Analysis of Curved Optical Waveguides by Conformal Transformation,” IEEE Journal of Quantum Electronics, Vols. QE-11, pp. 75-83, 1975, incorporated herein by reference, but performed in reverse.

Finally, the case of waveguide bending is the easiest to handle, since an equivalent conformal expression of the permittivity of a curved waveguide can be found in literature.

The broader implications of this result are also highly significant. While a primary focus of this approach is on optical applications, the method itself relies on general coupled mode theory, which can be applied to essentially any wave phenomenon. Since the derivation involves the well-known process of minimization, it can be extended in the same ways, such as through the incorporation of constraints using Lagrange multipliers.

Proper tapering is critical for all integrated photonics. Similarly, given the generality of the underlying proof, the approach described above is applicable to any phenomenon that can be described using coupled mode theory. This includes wave phenomenon in general, not only physical but abstract (such as traffic flow waves).

### Low Reflection Optical Couplers

Conventional grating designs often rely on partially-etched gratings or binary blazed gratings in order to enhance the coupling efficiency while attempting to reduce the reflected light. However, the impedance mismatch in such designs remains an issue. In some embodiments of the inventive approach, a metamaterial structure (tapers) is used to facilitate the adiabatic transition of the refractive index (n), resulting in an impedance matching grating. The inventive approach is effective in suppressing the reflections that frequently occur due to the impedance mismatch when coupling light into/out of a photonic chip through integrated optical couplers (I/O's). The inventive design is a combination of both binary gratings and metamaterial structures (tapers) that are optimized as described above.

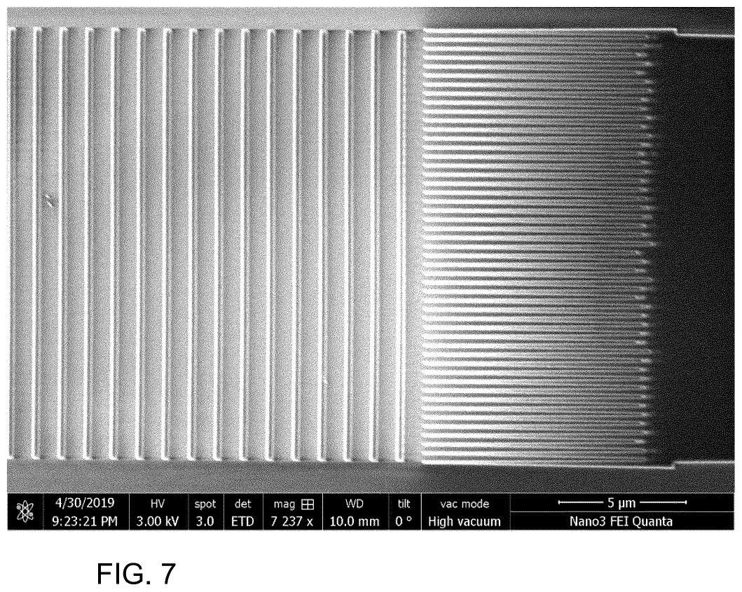

The working principle of the inventive scheme employs features of diffraction gratings, where uniform gratings are used to couple light into and/or out of a photonic chip. The key improvement involves the optimization of metamaterial structure (tapers) that were introduced/fabricated at the end of the binary gratings and at the interface of the waveguide as shown in FIG. 7.

The optimized tapers enhance the adiabatic transition of the refractive index (n), suppressing the reflections that frequently occur at the interface(s), as a result of the abrupt change in the refractive index. Furthermore, this transition benefits from the optimization of parameters as described in the adiabatic taper discussion above. The inventive approach is based on the mathematical relationship:

$$n ( x , y , z ) = \sqrt{\frac{{n}_{F}^{2} ( x , y ) - {n}_{I}^{2} ( x , y )}{{z}_{F} - {z}_{I}} ( z - {z}_{I} ) + {n}_{I}^{2} ( x , y )} , \qquad ( 50 )$$

where n is the index of refraction, I is the initial value of the individual metamaterial structure (tapers), and F is the final value of the individual metamaterial structure (tapers).

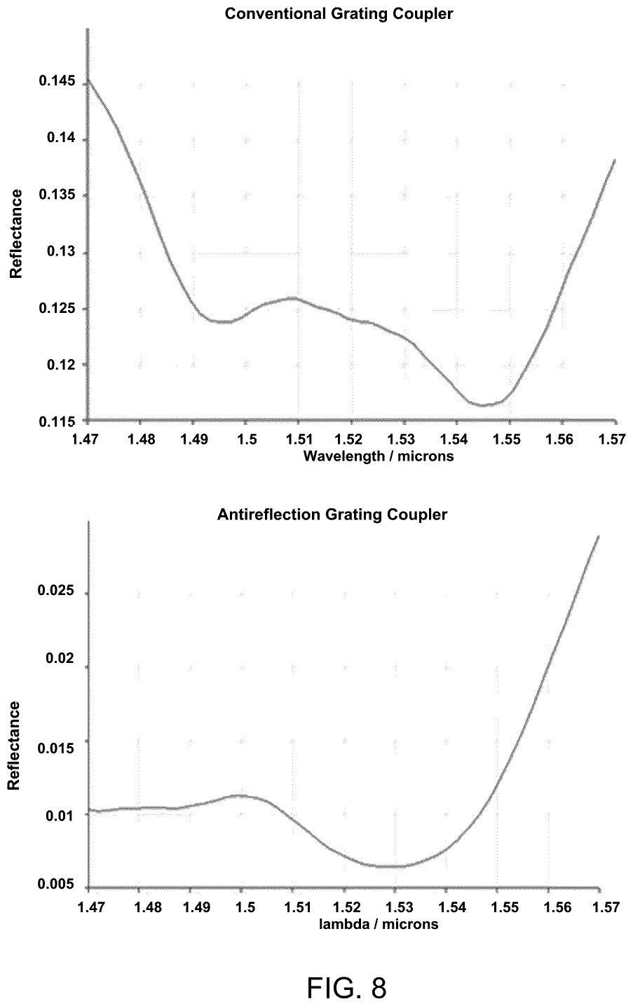

FIG. 8 provides plots of simulated spectral S parameters of optical I/O couplers with (lower) and without (upper) the inventive impedance matching structure.

When incorporating photonic chips into optical transmission assemblies, I/O couplers are essential elements in the majority of designs. The inventive couplers can be implemented in any photonic chip for any application (telecommunication, biomedical, geomorphic, remote sensing, etc.).

1. A method for determining axial resolution or depth in an optical imaging system configured for imaging an object having one or more surfaces, the method comprising:
scanning the one or more surfaces by projecting light from a tunable narrowband laser source into an interferometer to generate an interferogram while applying phase modulation to the projected light; and
applying multirate signal processing to the interferogram to determine positional information for the one or more surfaces of the object.

2. The method of claim 1, wherein applying phase modulation comprises inserting a signal generator into a sample arm of the interferometer to apply phase modulation that is slow compared to the time taken to measure a single frequency.

3. The method of claim 1, wherein applying phase modulation comprises inserting a signal generator immediately downstream of the laser source to apply fast modulation to increase a maximum unambiguous range, wherein the fast modulation repeats after every sweep frequency.

4. The method of claim 1, further comprising repeating scanning and applying for multiple iterations.

5. The method of claim 1, wherein applying multirate signal processing comprises defining multiple channels within the interferogram and combining the multiple channels in a frequency domain to increase time domain resolution.

6. The method of claim 1, wherein applying multirate signal processing comprises defining multiple channels within the interferogram and interleaving the multiple channels to increase frequency resolution.

7-8. (canceled)

9. The method of claim 1, wherein the optical imaging system is a swept source optical coherence tomography (SS-OCT) system.

10. (canceled)

11. The method of claim 101, wherein the optical imaging coherence tomography system is a Light Detection and Ranging (LiDAR) system.

12.-16. (canceled)

17. An assembly for determining axial resolution or depth in an optical imaging system configured for imaging an object having one or more surfaces, the assembly comprising:
a tunable narrowband laser source;
an interferometer configured to generate an interferogram at a detector using light from the laser source;
a phase modulator inserted within an arm of the interferometer; and
a multirate filter bank configured for processing the interferogram to determine positional information for the object.

18. The assembly of claim 17, wherein the phase modulator is inserted into a sample arm of the interferometer, and wherein the phase modulator is configured to apply slow modulation to the light to improve axial resolution in a length domain.

19. The assembly of claim 18, wherein the optical imaging system is a swept source optical coherence tomography (SS-OCT) system.

20. The assembly of claim 17, wherein the phase modulator is inserted downstream of the laser source, and wherein the phase modulator is configured to apply fast modulation to the light to increase a maximum unambiguous range of detection.

21. The assembly of claim 20, wherein the optical imaging system is a Light Detection and Ranging (LiDAR) system.

22. The assembly of claim 17 wherein the multirate filter bank is configured to define multiple channels within the interferogram and combine the multiple channels in a frequency domain to increase time domain resolution.

23. The assembly of claim 22, wherein the optical imaging system is a swept source optical coherence tomography (SS-OCT) system.

24. The assembly of claim 17, wherein the multirate filter bank is configured to define multiple channels within the interferogram and interleave the multiple channels to increase frequency resolution.

25. The method of claim 24, wherein the optical imaging system is a Light Detection and Ranging (LiDAR) system.
